(19) United States
(12) Patent Application Publication
(10) Pub. No.: US 2007/0248542 A1
(43) Pub. Date: Oct. 25, 2007

(54) Devices and methods for individualized detection of nutrient imbalance via olfactory system

(75) Inventors: Edward Kane, Millville, NJ (US); Timothy Cunninghamm, Liberty Lake, WA (US)

Correspondence Address: LAW OFFICES OF KHALILIAN SIRA, LLC, 9100 PERSIMMON TREE ROAD, POTOMAC, MD, 20854, US

(73) Assignee: BodyBio, Inc., Millville, NJ (US)

(21) Appl. No.: 11/409,828

(22) Filed: Apr. 24, 2006

(51) Int. Cl. A61K 49/00; G06Q 50/00

(52) U.S. Cl. 424/9.1; 705/2

(57) ABSTRACT

A method of detecting an imbalance of nutrients in an individual is disclosed. The method comprising administration of a smell test, wherein the smell test comprises one or more support members and one or more nutrients that are aroma-intensified and incorporated into one or more support members, the method further comprising the steps of: (a) designating a number for each nutrient to create a nutrient list; (b) designating a score system that allocates numerical values to describe the smell associated with each nutrient; (c) smelling the nutrient to obtain a registered smell; (d) allocating a score to the registered smell of the nutrient; (e) identifying the nutrient by consulting the nutrient list of step (a); and (f) tabulating and calculating numerical values obtained for each nutrient to obtain information on balance, surplus or deficiency of the one or more nutrients.

### I. FIELD OF THE INVENTION

The present invention relates to devices and methods for providing personalized nutrient information. More particularly, the invention relates to devices and methods for detecting nutrient imbalance in an individual through the use of the olfactory system.

### II. BACKGROUND OF THE INVENTION

Taste and smell are two prominent indicia in the scientific exploration of our innate sensory relationships to the world as they are the oldest senses in the primary cluster of touch, sight and hearing. Together taste and smell are the gatekeepers of the biomass that feeds, nourishes and balances all other senses and responses to life. The olfactory system, which senses and processes thousands of odors, is one of the oldest and most vital parts of the brain. For most animals, it is the primary mode of communication and influences many important functions, including reproduction and taste.

The analysis of olfactory system on the molecular level has only recently been seriously pursued. A large gene family coding for odor binding sites, or receptors, has been identified in the olfactory lining of the nose. In the olfactory bulb (a brain structure just above the nose) information from these receptors is organized into patterns that the brain may interpret as different odors. The nose contains specialized sensory nerve cells, or neurons, with hair-like fibers called cilia on one end. Each neuron sends a nerve fiber called an axon to the olfactory bulb. Most animals can distinguish thousands of odors. Early studies showed that the different olfactory neurons response to different odors stimulates specific patterns of activity in the olfactory bulb.

Recently, researchers identified a surprisingly large family of genes in rats that appears to code for odor receptors. This gene family is one of the largest ever discovered, programming 500 to 1,000 different types of receptors. It has been hypothesized that this large and diverse group of genes helps animals detect a huge variety of odors. In rats and mice, the olfactory lining is divided into four zones, each containing neurons with different odor receptors. Neurons expressing the same receptor genes within each zone appear to be randomly arranged.

Research suggests that an individual odor molecule stimulates several types of receptors, each of which responds to a part of the molecule's structure. Brain mapping techniques have shown that the pattern of glomeruli activated by each odor forms a map or code that the brain may recognize as a unique scent.

In mammals, odorants are inhaled through the nose where they contact the olfactory epithelium. Olfactory receptor neurons in the olfactory epithelium transduce molecular features of the odorants into electrical signals which then travel along the olfactory nerve into the olfactory bulb. Axons from the olfactory sensory neurons converge in the olfactory bulb to form glomeruli (singular glomerulus). Inside the glomulerus, the axons contact the dendrites of mitral cells and several other types of cells. Mitral cells send their axons to a number of brain areas, including the piriform cortex, the medial amygdala, and the entorhinal cortex. The piriform cortex is probably the area most closely associated with identifying the odor. The medial amygdala is involved in social functions such as mating and the recognition of animals of the same species. The entorhinal cortex is associated with memory. The exact functions of these higher areas are a matter of constant scientific research and debate.

Proteins, found in the olfactory mucus, have recently been discovered that bind to odorants. These have been termed the Odorant Binding Proteins (OBPs). Odorants dissolve in the aqueous/lipid environment of the mucus and then bind to an OBP. It is thought that these proteins facilitate the transfer of lipophilic ligands (odorants) across the mucus layer to the receptors, and also increase the concentration of the odorants in the layer, relative to air. There are two other proposed roles for these proteins as, (1) a transporter, in which they would bind to a receptor with the ligand and accompany it across the membrane and (2) as a terminator, causing “used” odorants to be taken away for degradation, allowing another molecule to interact with the receptor. The protein could also be acting as a kind of protector for the receptor, preventing excessive amounts of odorant from reaching the receptor.

It appears that there may be hundreds of odorant receptors, but only one (or at most a few) is expressed in each olfactory receptor neuron. A large family of odorant receptors was cloned in 1991 by Linda Buck and Richard Axel, (Cell 5:65(1), 175-87 (1991)), and the mRNA encoding these proteins has been found in olfactory tissue. These families may be encoded by as many as 1000 different genes. This is a huge amount and accounts for about 2% of the human genome. In humans, however, most are inactive pseudogenes and only around 350 codes for functional receptors. Glutamate has been proposed as the olfactory cell neurotransmitter in turtle, toad and in rat—mediating transmission at the first synapse in the pathway (olfactory receptor neuron (ORN)-mitral cell). There is evidence that noradrenaline is a neurotransmitter in the rat olfactory bulb. There is considerable clinical interest in this system because of the number of conditions associated with diminished noradrenaline activity in which olfactory discrimination is also impaired, including Korsakoff's disease, normal ageing, Parkinson's disease and Alzheimer's disease. Both behavioral and molecular studies point to a potentially important role of dopamine in olfaction. Parkinson's patients, who have reduced dopamine levels, also have impaired odour recognition. Injection of dopamine analogues reduces olfactory sensitivity in rats. Dopamine may play an important neuormodulatory role in olfaction by reducing transmitter release from ORNs.

The olfactory and gustatory systems are both chemosensory senses because both transduce chemical signals into perception. The olfactory system must accomplish several tasks (e.g., create a representation of the odor, determine the concentration of the odor, distinguish a new odor from the background environmental odors, identify the odor across different concentrations, and pair the odor with a memory of what the odor represents. To accomplish all of these functions, the olfactory system uses many areas of the brain. Representations of the odor may be encoded by space (a pattern of activated neurons across a given olfactory region corresponds to the odor), time (a pattern of action potentials by multiple neurons corresponds to the odor) or a combination of the two. Scientists debate whether the odor code is primarily temporal or spatial.

Olfactory neurons in the primate orbitofrontal cortex decrease their responses to a food eaten to satiety, but remain responsive to other foods, thus contributing to a mechanism for olfactory sensory-specific satiety. It has been shown in neuroimaging studies that the human orbitofrontal cortex provides a representation of the pleasantness of odor, in that the activation produced by the odor of a food eaten to satiety decreases relative to another food-related odor not eaten in the meal. In the same general area there is a representation of the pleasantness of the smell, taste and texture of a whole food, in that activation in this area decreases to a food eaten to satiety, but not to a food that has not been eaten in the meal. See, for example, Rolls E T. Chem Senses 26(5):595-604 (2001). Zinc taste test in pregnant women has been found to be well correlated with serum zinc level and provided a fair idea of zinc deficiency. See, for example, Garg et al., Indian J Physiol Pharmacol. 37(4):318-22 (1993).

In another study, the latent-learning paradigm was used to examine whether replete rats can recognize sodium and calcium and whether they use that knowledge to guide consumption when subsequently mineral deprived. The results of the study demonstrated that that there is an evidence for the existence of innate calcium and sodium appetites in calcium-deprived rats. They indicate that these distinct appetites are centrally generated behaviors and are not simply due to peripheral alterations in taste perception. See, for example, Coldwell et al., Am J Physiol. 265:1480-1484 (1993). In the context of taste component analysis of mixtures, it has been found that the rodent taste system can specifically respond to sodium chloride in a sodium chloride-sucrose mixture. Mineral taste test and its correlation with serum mineral level and satiety have been studied in mammals for several minerals such as iron, (see, for example, Woods et al., Physiol. Behav. 19(5):623-6 (1977), magnesium (see, for example, McCaughey et al., Appetite. 38(1):29-38. (2002).

In order to evaluate the emotional reactivity associated with each primary taste, sweet, salty, sour and bitter tastes were evaluated through analysis of the variations of autonomic nervous system (ANS) parameters. Rousmans et al. Chem. Senses, 25(6):709-718 (2000). The hedonic dimension of the taste sensation was found to play a crucial role in the control of many taste-mediated responses related to food ingestion or rejection. Results of the study evidenced a significant effect of primary taste on skin resistance amplitude, skin temperature amplitude, skin blood flow amplitude, and instantaneous heart rate increase. The four primary tastes could be associated with significantly different ANS responses. The pleasantly connoted and innate-accepted sweet taste induced the weakest ANS responses whereas the unpleasant connoted tastes (salty, sour and bitter) induced stronger ANS responses, the innate-rejected bitter taste inducing the strongest ones.

The use of olfactory system to test for certain diseases or disorders has been reported previously. For example, U.S. Pat. No. 6,957,038 discloses a self-scoring test kit and method for early self-screening of Alzheimer's disease by detection of diminished olfactory function. The test kit is comprised of a plurality of pages attached to each page via adhesive is a microencapsulated strip that, when scratched, releases a different, distinct odor.

U.S. Pat. No. 6,132,830 discloses a smell test kit for measuring the sense of smell of a test subject. The smell test kit comprises a set of cards, a set of fragrance strips, adhesives, and a plurality of releasable microcapsules. The plurality of releasable microcapsules is contained within the adhesive which secures at least a portion of the fragrance strips to the cards. When the adhesive is overcome and the fragrance strips are removed from the cards, the releasable microcapsules burst open and emit a distinct scent for each of the cards. This patent tests the brains mental acuity via the olfactory system testing for the purpose of determining the brain and olfactory system structural integrity.

The prior art does not provide a method for detection of nutrient imbalance through microencapsulation technology and the olfactory system. While there may be products in the marketplace that offer vitamin supplements in bottles that can be detected by smell, these products are not user friendly and does facilitate repeat use. For example, U.S. Patent Application No. 2005/0028829 discloses methods and products for detecting vitamin imbalance in an individual through the use of olfactory system and a series of bottles containing vitamins. Individual test taker must determine a need for a vitamin supplement through a score of 1-10. The products disclosed in this application does not allow for rapid and accurate testing of vitamins or other nutrients. The shelf life of the vitamins is inevitably short due to repeat opening and closing of the bottles, which in turn diminishes any aroma associated with these vitamins. The high costs of these products ($200 per set of 20 bottles), in addition to the difficulty in handling and transporting them, make these products unattractive by modern consumers.

What is desirable, then, is an individualized nutritional kit or device that provides effective, economical, and accurate results and facilitates diagnosis of nutritional imbalance in the individual. For consumers, it is important to acquire accurate information tailored to their specific nutritional needs through a test and device that are simple to use, yet efficient. The invention described herein addresses this and other needs by allowing consumers to acquire knowledge about their own personal nutritional needs and assist them to maintain a balanced nutritional supplementation regimen.

### III. SUMMARY OF THE INVENTION

The invention as described herein provides methods, devices and kits for detecting an imbalance of nutrients in an individual comprising administration of a smell test, wherein the smell test comprises one or more support members and one or more nutrients that are aroma-intensified and incorporated into one or more support members, the method comprising the steps of: (a) listing nutrients and designating a number for each nutrient to create a nutrient list; (b) designating a score system that allocates numerical values to describe a smell associated with each nutrient; (c) smelling the nutrient to obtain a registered smell; (d) allocating a score to the registered smell of the nutrient; (e) identifying the nutrient by consulting the nutrient list of step (a); and (f) tabulating and calculating numerical values obtained for each nutrient to obtain information on balance, surplus or deficiency of the one or more nutrients, wherein the administration of the smell test detects the imbalance of nutrients in the individual.

In one embodiment, the method further comprises the steps of: (g) analyzing the significance of results obtained in step (f); and (h) extrapolating the results of the analysis to identify a specific pattern for long term or short term nutritional needs and counter-indication of specific nutrients and/or drugs for the individual. The analysis of results includes statistical analysis of the scores through calculating the mean, standard deviation, and variance of scores.

In another embodiment the one or more nutrients are aroma-intensified through a micro-encapsulation technology and the smell test is achieved through scratch and sniff, or removal of a strip from the micro-encapsulated nutrient. The strip can either be a completely separate piece such as a piece of paper, or a portion of the support member which is folded back on itself.

In one embodiment, a list of nutrients is located in a removably covered answer key that correctly identifies each of the aroma containing nutrients.

The one or more support members of the device of the invention include any media that can incorporate or host an aroma-intensified nutrient and include any synthetic or natural material, such as for example, paper, plastic, rubber, metal, fiber, cotton, glass, or a combination thereof among others. In one embodiment, the support members are made of paper that includes, by way of example and not limitation, post cards, books, magazines, single pin cards, scratch offs, pull tabs, card scan, game cards, phone cards, gift cards, and internet access cards, among others.

In one embodiment, the books and magazines are related to children, health, fashion, sport, cars, house decoration, food, fiction, and entertainment, among others.

In another embodiment, the support member comprises a punched-hole book, a ring type book, a bound book, and an address book, among others.

In yet another embodiment, the one or more support members comprise the internet, intranet, or an equivalent thereof.

In another embodiment, the method additionally includes a description of several nutrients tested, and a list of indicated and counter indicated nutrients and/or drugs.

In one embodiment, the score system comprises scores of, 1-2, 1-3, 1-4, 1-5, 1-6, 1-7, 1-8, 1-9, or 1-10. In a preferred embodiment, the score system comprises scores 1-7 that designates numerical values to desirable, pleasant, no taste, taste something, taste something not so pleasant, dislike, and awful, respectively.

A wide variety of nutrients are encompassed within the scope of the invention. The nutrients including, by way of example and not limitation, vitamins, precursors of vitamins, oils (including essential fatty acids, (EFA's) and non-essential oils), minerals, enzymes, co-enzymes, and amino acids, among others. Nutrients also include analogues, variants, biological equivalents, isomers, optical isomers (e.g., D and L forms), functionalized forms, substituted forms, salts (e.g., citrates, lactates, gluconates, fumarates, esters, acetylated forms, partial acetylated forms, etc., among others), metabolites, intermediates, chelated forms, buffered forms, alkaline and/or basic forms, or a combination thereof, among others.

In one embodiment, the smell test is conducted on nutrients on a pre-determined order. The order of nutrients tested is selected on the basis of the chemical structure of the nutrient, the pH of the nutrient, the intensity of the aroma and the type of aroma generally associated with the nutrient, or a combination thereof. The type of aroma associated with nutrients includes sweet, bitter, tangy, spicy, woody, rain forest smell, fishy, earthy, and floral, among others.

The method of the invention is administered to healthy individual, or symptomatic and/or asymptomatic patients. The nutritional information achieved through the smell test of the invention is provided for the general well being, or treatment and/or prevention of a disease or disorder in an individual.

In another aspect, the invention provides for a smell test kit for detection of a deficiency or surplus of one or more nutrients in an individual via the olfactory system of the individual comprising one or more support members and a plurality of aroma-intensified nutrients incorporated into the one or more support members, the smell test kit further comprising: (a) a list of numbers designated for each nutrient; (b) a table that allocates numerical values to a description of aroma perceived by a test taker after the nutrient was smelled; (d) an instruction to use the test; and (e) an explanation of the results of the test. The smell test kit may additionally contain (f) a first section for statistical analysis of the significance of the result; and (h) a second section for extrapolating results of the analysis to identify a specific pattern for long term or short term nutritional needs and counter-indication of specific nutrients and/or drugs for the individual.

In yet another aspect, the invention provides for a nutritional analysis and recommendation book containing a nutrient smell test comprising: (a) a multiplicity of nutrients micro-encapsulated into the book; wherein an aroma associated with the nutrients is smelled through a scratch and sniff test or a removal of a strip from the nutrients; (b) a chart that designates a number for each of the nutrients; (c) a table that allocates numerical values to a description of aroma perceived by a test taker after the nutrient was smelled; (d) a first section for tabulating and calculating numerical values obtained in step (c) in order to obtain individualized nutritional recommendations; (e) a second section that provides analysis of significance of results obtained by individual test taker; and (f) a third section that provides long term and short term nutritional recommendations and counter-indication advice on nutrients or drugs on the basis of results obtained in the first and the second sections.

Other preferred embodiments of the invention will be apparent to one of ordinary skill in the art in light of what is known in the art, in light of the following description of the invention, and in light of the claims.

### V. DETAILED DESCRIPTION OF THE INVENTION

Definitions

As used herein “nutrients” are defined broadly as organic or inorganic compounds or molecules that either alone or in combination with an aromatic or non-aromatic agent produce a specific sent or aroma. Nutrients encompass macronutrients and micronutrients that are present in either an isolated and purified form or in a crude form as part of a food or food product along with other molecules and compounds.

Nutrients include, by way of example and not limitation, any synthetic and nor natural organic and inorganic compounds and molecules including, vitamins, precursors of vitamins, oils (including essential fatty acids, (EFA's) and non-essential oils), minerals, enzymes, co-enzymes, and amino acids, among others. Nutrients also include analogues, variants, biological equivalents, isomers, optical isomers (e.g., D and L forms), functionalized forms, substituted forms, salts (e.g., citrates, lactates, gluconates, fumarates, esters, acetylated forms, partial acetylated forms, etc., among others), metabolites, intermediates, chelated forms, buffered forms, alkaline and/or basic forms, or a combination thereof, among others.

The terms “amino acids”, as used herein, refers to oligopeptides, peptides, polypeptides, or proteins, a fragment of any of these, and to naturally occurring or synthetic molecules.

The term “variant,” as it used herein, refers to alternative forms of the nutrients, whose structure or function may or may not be altered. Common mutational changes which give rise to variants are generally ascribed to natural deletions, additions, or substitutions of amino acids. Each of these types of changes may occur alone, or in combination with the others, one or more times in a given amino acid molecule.

As used herein “aroma intensified nutrients” are nutrients that have been subjected to an aroma-intensifying process, such as, for example, micro-encapsulation.

The invention as disclosed herein provides nutritional detection devices and methods that provide a framework for analyzing the individual consumer's nutritional imbalance that is specific to that individual and specifically addressing consumer's needs for supplements. The method and devices of the invention provides for therapeutic regimens that are tailored to take account of the vast differences that exist in the biological make up of human beings and other external and internal factors that call for modification of nutrient supplementations from time to time. A nutritional regimen that would be beneficial for an individual may prove to be poisonous and harmful for another based upon the physiological, immunological, biochemical, and/or genetic differences that exist among people.

The methods and devices of the invention are tailored to detect the nutritional requirement of the individual, while accommodating for gender and race differences, as well as periodic or constant or continuous changes that occur within the body of the individual. The human body responds differently to different internal and external changes. These changes are brought about by, for example, ailments, ageing, pregnancy, menopause, age, occupation, life-style, different seasons and weather conditions (e.g., light, humidity, draught, cold, heat, air pressure, etc), cyclical physiological changes (e.g., menstruation cycle in women), among others. The ease of operation of methods and devices of the invention provides for periodic (e.g., daily, weakly, monthly, seasonally, annually, etc) detection of the individual's nutritional imbalances and effective modification of the supplementation therapy on a periodic basis.

1. Nutrient Support Members

The device of the invention includes one or more support members that contain at least one nutrient that has an aroma or a sent capable of detection by normal olfactory system of a consumer. The support members are connected or disconnected from one another. The nutrients are coated onto or encapsulated into the support member.

The nutrient support members of the invention are made of a variety of natural or synthetic materials including by way of example and not limitation, paper, plastic, rubber, metal, fiber, cotton, glass, or a combination thereof among others. In one embodiment, the support members are paper-based.

The nutrients are incorporated into varieties of support members including by way of example and not limitation, post cards, books, magazines (children, health, fashion, sport, cars, house decoration, food, fiction, entertainment-related books and/or magazines), stack of cards attached at one end (single pin), scratch offs, pull tabs, card scan, game cards, phone cards, gift cards, and internet access cards, among others.

In one embodiment, the nutrient support is a book. The nutrients are encapsulated in one or more pages of the book. The book contains instructions to use the nutrient test, including a description of several nutrients tested, the way that the test should be conducted and the procedure to score the test. Also included in the book, is a list of indicated and counter indicated nutrients and/or drugs in accordance with the result of the test. The book is in variety of shapes and bindings, including punched hole, ring type, address book, continuous ring type, etc, among others.

In another embodiment, the present invention is implemented on the Internet.

The technology underlying scratch & sniff web pages has been launched in the past few years. Internet sites implement scratch and sniff technology to induce and stimulate olfactory systems to smell a particular aroma of natural or synthetic product. This new internet sensation is hitting literally thousands of internet surfers daily. Typically, the screen provides a picture of a food being rich in certain vitamins or supplements (e.g., bread being rich in vitamin B). The consumer takes the mouse and place the cursor over the bread and then briskly move the cursor over the image (scratch it) for, for example, 20 seconds. After this time the cursor is placed over the item and the left mouse button is clicked. The next web page will have a visual image that will trigger a sense that the consumer is smelling bread. See, for example, Solid Alliance (Japan) MP3 players with cases emitting various food smells. The ICool MP3 Player (256 MB) uses six different smells: chocolate, rose, strawberry, lemon, cherry and blueberry, compatible with both Windows and Mac. Other scents that have already been incorporated in the internet sites include licorice, clove, peppermint, cherry, lemon, and vanilla. The methods of the invention can be implemented through internet using the aforementioned technologies.

2. Aroma-Intensified Nutrients

The nutrients tested in the smell test of the invention have preferably been processed through an aroma-intensification technique. Any aroma-intensification technology that results in condensation and intensification of aroma within a nutrient is encompassed within the scope of the invention. A preferred aroma-intensification process is micro-encapsulation. Micro-encapsulation achieves nutrients with intensified aroma that are essential in the success of the smell test.

Aroma, to begin with the basics, is a matter of molecules dislodging themselves from a substance's surface and finding their way into the nose. To achieve something that smells on paper, its molecules must be distilled into a sort of perfume (i.e., a highly volatile liquid) that is insoluble in water. This is brought about by emulsification of the liquid, which essentially means that the liquid has been sheared to a very high degree. Since oil and water do not mix, the finely sheared liquid will result in a solution containing millions of micro bubbles of essence suspended in liquid. The bubbles may then be conveyed into a plastic compound, which can then be used like ink and printed onto (preferably) some sort of stiff card stock with a modified printing press. When the plastic carrier dries, the bubbles of liquid are trapped inside until the card is scratched, whereupon they break open and become aromatic.

In one embodiment the aroma is released from micro-encapsulated nutrients by the use of scratch and sniff technology. Scratch and sniff stickers have been used for many years in different media, such as for example, children books and the occasional perfume strip in magazines. The long shelf life of the scratch and sniff stickers (the scratch and sniff works even in old books of 20 years or older) make them particularly useful for the administration of the nutrient smell test of the invention. The reason for the long shelf life of the sticker is due to the micro-encapsulation technology used to create them.

The basic idea behind scratch-and-sniff is to take the aroma-generating chemical and encapsulate it in gelatin or plastic spheres that are very small, on the order of a few microns in diameter. When the sticker is scratched, some of the spheres are ruptured and the smell is released. The smell is essentially held in millions of tiny spheres, and a few of the spheres are broken every time the sticker is scratched. The tiny spheres preserve the fragrance for years.

In another embodiment, the aroma is released from the micro-encapsulated nutrients by simply removing an adhesive tape. Aroma-intensified nutrients are adhered to a removable strip by adhesive means. In a preferred embodiment, the support members and the removable strip are made of paper. The nutrient smell test is administered by peeling or tearing away the strip from the micro-encapsulated nutrient. The strip can either be a completely separate piece of paper, or a portion of the support member which is folded back on itself. The manner of adhering a strip to a micro-encapsulated agent is disclosed in U.S. Pat. Nos. 4,988,557; 4,889,755; 4,661,388; and 4,606,956, the contents of all of which are incorporated herein by their entirety.

3. Nutrients

The invention disclosed herein provides methods and devices to detect and monitor individual's need for supplements periodically. A consumer smells the nutrients to determine the need for supplementation or avoidance of the nutrient. Smelling food to determine need is germane to the survival of species, a fundamental tool and basic instinct in the animal kingdom to determine whether there is a need for a particular food. The olfactory system not only dictates what is safe to eat but also guides us to foods that meet our individual nutritional needs. For example, a person who prefers or craves eggs may need the unique combination of zinc, amino acids, phosphatidylcholine and other nutrients found in eggs.

Nutrients are broadly defined within the scope of the invention and include essential substances such as vitamins and minerals. Vitamin and mineral deficiencies cause mild to serious diseases and disorders. It is to be noted that excessive amount of supplements creates more problems than their deficiencies, leading to toxic levels in the body. Accordingly, in order to maintain or enhance an individual's health, it is imperative to know the balanced level of vitamins and/or minerals specific to the individual.

Each individual has a unique level of required vitamins and supplements. A vast majority of Americans take vitamin and mineral supplements as recommended by the American Medical Association, in order to help meet their daily nutritional needs and fend off illness and disease. The problem lies in that recommended dosage of vitamins and minerals do not take into account of the differences among the individual's for their nutritional needs. A person taking a multivitamin over time will inevitably take too much of certain nutrients and not enough of others. The U.S. Government sets allowances for most vitamins and minerals based on scientific studies of how much of each nutrient men, women and children use up on a daily basis. For example, the daily recommend amount of calcium is 1000 mg. for adults ages 19-30. A person who has a diet rich in milk, cheese and yogurt obtains more than this amount each day and probably needs no additional supplementation. Administration of calcium supplement to this person may result in over concentration of calcium, which over time leads to inhibition of absorption of iron, zinc and other minerals.

3.1 Vitamins

Vitamins are very important for small functions in the body. Vitamin supplements are advantageous because they help those who do not obtain enough vitamins from their food. In contrast to those who require vitamin supplements in order to make up for their diets that lack the required nutrition, there are some people that already have a well balanced diet and no vitamin deficiencies. It is thought that if these people intake vitamin supplements they would exceed the total content of vitamins in their bodies, which is harmful to their health. Vitamins in excess are not beneficial to the body. Vitamin overdose can potentially become a problem for those who do not determine their individualized requirement for specific vitamins and are of the misguided belief that more vitamins they consume the better their health may be. An example of harmful effect of vitamin over supplementation is the excess use of vitamin E, which has proven to be harmful and fatal.

In general, vitamins are classified into water- or fat-soluble. The water-soluble acidic vitamins such as nicotinic acid and vitamin C possess an acidic function and their concentration can be analyzed using CE with high pH borate or phosphate buffers. However, the fat-soluble vitamins such as vitamins A and E are neutral and have poor water-solubility and require use of a chromatographic-based method.

The nutrient smell test of the invention includes a wide variety of vitamins as the nutrient. Vitamins encompassed within the scope of the invention includes, for example, natural or synthetic vitamins, analogues, variants, biological equivalents, isomers, optical isomers (e.g., D and L forms), functionalized forms, substituted forms, salts (e.g., citrates, lactates, gluconates, fumarates, esters, acetylated forms, partial acetylated forms), metabolites, intermediates, chelated forms, buffered forms, alkaline and/or basic forms of the vitamins, among others.

The vitamins or vitamin pre-cursors used within the scope of the invention include, by way of example and not limitation, vitamin A, vitamin B-1, vitamin B-2, vitamin B-6, vitamin B-12, vitamin C, vitamin D, vitamin E (e.g., alpha-tocopherol or gamma-tocopherol forms of vitamin E, among other forms), Biotin, choline, calcium lactate pentahydrate, folic acid, betaine hydrochloride/pepsin, beta-carotene, inositol, niacin, para amino benzoic Acid (PABA), and pantothenic acid, among others. The vitamins according to the invention include all natural and/or synthetic forms of the vitamins, different bio-available forms, bioequivalent, and metabolites of the vitamins, among others. A brief description of certain vitamins and effect of their deficiency or surplus in the body is detailed below.

Pantothenic Acid (vitamin B5) is required for the synthesis of certain steroids produced in the adrenal glands via the action of coenzyme A. The recommended concentration for adults is 5 mg but the optimal daily intake may be 100 mg. The average intake of teenagers is about 5 mg per day. The best food sources for this vitamin are brewer's yeast, wheat germ, wheat bran, royal jelly, whole-grain breads, cereals, green vegetables, peas, beans, peanuts, crude molasses, liver and egg yolk. The highest levels are found in beef liver at 4.8 mg per 3 ounces.

Pantothenic Acid is water-soluble and stable in moist heat, but unstable in dry heat and acid or basic pH situations. Little is lost during normal cooking but 50% loss occurs in vegetables when they are frozen and 65% loss when they are canned. In addition, processed and refined grains lose about 50%, while processed meats lose up to 70% of vitamin B5. Pantothenic acid is readily absorbed from the gastrointestinal tract. It is stored in high amounts in the adrenal glands but about 70% of absorbed pantothenic acid is excreted in the urine. Before pantothenic acid is utilized it must first be converted to the sulfur-containing pantotheine. Pantotheine is currently fairly expensive and should be used only in select cases.

A serious deficiency of pantothenic acid is uncommon because of its wide distribution in foods. Experimentally-induced human vitamin B5 deficiency has caused insomnia, leg cramps, paresthesias of the hands and feet, mental depression, decreased antibody formation, easy fatigue, gastrointestinal disturbance, and upper respiratory infections.

The scientific data indicate that pantothenic acid compounds (calcium or sodium pantothenate, panthenol) at very high doses (approximately 10,000 mg per day in some cases for a number of years) may result in acute or chronic toxic effects such as long term diarrhea and gastrointestinal disturbances.

Beta-carotene is the molecule that gives carrots their orange color. It is part of a family of chemicals called the carotenoids, which are found in many fruit and vegetables, as well as some animal products such as egg yolks. Carotenoids were first isolated in the early 19th century, and have been synthesized for use as food colorings since the 1950s. Biologically, beta-carotene is most important as the precursor of vitamin A. It also has anti-oxidant properties and may help in preventing cancer and other diseases. In the last few years increasing numbers of reports have suggested that the use of beta carotene may act to prevent the development of various malignancies. Beta Carotene and other similar compounds have differentiating properties that appear to affect cell growth and maturation. Scientific reports have indicated that beta carotene is less toxic to the liver in high doses as compared with to Vitamin A. Large doses of beta carotene will increase the body's demands for Vitamin E; therefore, those who consume 50,000 to 100,000 units of beta carotene per day will need to also increase the intake of vitamin E to 1,000 to 2,000 units per day. The definitive mode of interaction between beta carotene and vitamin E is yet to be understood. In most individual's there would be no need to supplement beta carotene if the diet is rich in vitamin A or carrot juice. Beta carotene, like vitamin A and E is stored in the liver. The effect of beta carton in the prevention of cervical cancer, lung cancer, breast cancer and colon cancer, and melanoma is under investigation.

Vitamin E is an important anti-oxidant. It acts as a free radical scavenger to prevent the byproducts of chemical-cell interaction to cause cell damage. Free radicals are likely responsible for all or most of the degenerative diseases e.g. arthritis, heart disease, cancer, senility etc. The absorption or scavenging of free radicals would protect our cells from this type of injury. Other free radical scavengers include zinc, vitamin C, and selenium.

Studies have reported that vitamin E protects against some of the toxicities of ionizing radiation. Vitamin E may help to decrease the toxicity of certain chemotherapy drugs. It is also reported that vitamin E may decrease some of the harmful effects of solar radiation on the skin. Vitamin E appears to have stabilizing effect on the vascular system and is useful in decreasing menopausal and premenstrual symptoms. Vitamin E can also be used in lotions or creams to protect the skin or to treat for burns and especially to treat burns secondary to radiation therapy. This vitamin has also proved useful in patients with dermatitis resulting from poor blood circulation. Over supplementation and surplus of vitamin E is reported to be harmful and toxic.

Vitamin B complexes are important membrane stabilizers. They are natural tranquilizers or anti-stress vitamins because of this property. They are important vitamins to help the nerve function and the nerve cell regeneration. The B complex vitamins contain agents that important in protecting the skin against the harmful effects of Ultra-violet radiation. These agents are excellent in UV screening and help to protect against the development of skin cancer or sun-induced skin damage e.g. wrinkling and solar keratoses. It is known that fair complexioned people are more sensitive to sun and are at a greater risk for the development of skin cancers of all types. It is also known that women using oral contraceptives increase their utilization of the B vitamins and need to supplement their diet with vitamin B complex. Individualized under high stress may also need a higher dose of vitamin B complex. The B vitamins are water soluble and unlike beta carotene and vitamin E, vitamin B complexes are not stored in the body and must be taken with food. The use of vitamin B complex in a dose of 100 mg once to three times a day with meals has been recommended per individual's tolerance. Over supplementation of this vitamin may cause renal or liver disfunction.

Vitamin C is important for tissue healing. It is reported that patients with peptic ulcers will heal faster on vitamin C supplements than those without extra vitamin C. Vitamin C is an anti-oxidant. It is also water soluble like vitamin B complex. Some physicians advise that vitamin C be used in doses of 2,000 to 6,000 mg after each meal to take advantage of its anti-oxidant effects. Side-effects of large doses, however, include diarrhea and flatulence. Additionally, some studies have shown that a reduction in the number of colon polyps in individuals taking high doses of vitamin C. Such polyps are precursors for colon cancer. In these cases, physicians recommend the use of vitamin C with a low fat diet and high dietary fiber. Alcohol consumption has been reported to increase the excretion of vitamin C and depleting all of the water soluble vitamins. High dose of vitamin C may be a problem in patients with a tendency to kidney stone formation. In patients receiving iron supplements to help treat iron deficiency, the use of vitamin C taken at the same time as the iron medicine will greatly enhance the absorption of iron. Vitamin C helps with wound healing and healing of burns, and it improves the strength of the walls of the blood vessels, which may help to reduce the easy bruising seen with some individuals.

Accordingly, it is concluded that a balanced level of vitamins is a critical factor in the health and well being of individuals. The methods and devices of the invention described herein facilitate obtaining a well balanced vitamin supplementation regimen that is tailored to the specific nutritional requirement of the individual test taker.

3.2 Minerals

Overall, all life forms are nutritionally dependant in minerals. Mammals are capable of choosing by taste the appropriate levels for supplementation of minerals. The voluntary intake of five calcium salts and eight mineral chlorides by male rats was assessed in one study. The result indicated that all minerals have some taste inference, and may make water taste better. The results illustrated that, as is the case for sodium, rats spontaneously ingest low concentrations of calcium and several other mineral solutions in preference to water alone. These studies demonstrate that the lower the cation's ionic charge, the greater the intake and higher the most accepted concentration of minerals. The adult rat pattern of discrimination between the salts emerges between 3 and 18 days of age. Subsequently, the preference for sodium over the other salts increases into adulthood. See, for example, Dev. Psychobiol. 27(6):381-94(1994).

Twenty five elements are recognized as the “bioessential” components of plants, animals and man. Carbon (C), Nitrogen (N), Oxygen (O) and Hydrogen (H) are primarily derived from air and water and are not important as supplements. Of the remaining minerals found in the soils, Pottasium (K), Phosphorus (Ph), Sulphur (S), are considered macronutrients. While Calcium (Ca) and Magnesium (Mg) are needed in relatively large amounts, the rest, Boron (B), Copper (Cu), Chloride (Cl), Iron (Fe), Manganese (Mn), Sodium (Na), Zinc (Zn), and Molybdenum (Mo) are considered micronutrients, and are the most important and best understood. Cobalt and the rare earth elements are also included as nutrients although information on their roles in the body is less exact.

Also included within the scope of are the salts, chelated forms, and buffered, basic and/or alkaline forms of the minerals, among other forms. For example, chloride can be used as ammonium chloride, calcium can be used as calcium citrate, calcium carbonate, calcium gluconate, calcium lactate, calcium chloride, calcium malate, calcium aspartate, calcium ascorbate, among others. A brief description of certain minerals is disclosed below.

Selenium is an anti-oxidant with activity as a free radical scavenger. Populations with high blood selenium levels are found to have lower death rates due to cancer. Cancer patients have low selenium blood levels. Selenium works in harmony with vitamin E. Selenium toxicity, however, can occur and the dose of selenium higher than 100 microgram per day has been reported to cause toxicity.

Calcium is believed to be deficient in most diets. This deficiency is most prevalent in women after menopause. Calcium consumption through a balanced diet along with exercise is the best means to prevent calcium deficiency. Once calcium is lost and signs of osteoporosis develop the medical problem may be hard to reverse. Collapse of bone and fractures commonly of the pelvis may occur in such individuals. Calcium, like B complex, acts as a membrane stabilizer and natural tranquilizer as does magnesium and potassium. Calcium and magnesium have been administered together in a ratio of 2:1, with a daily intake of Calcium of 1,000 to 1,500 mg reported to be reasonable.

Magnesium in the form of magnesium oxide in combination with vitamin B-6 has been shown to dissolve certain types of kidney stones (calcium oxalate stones). Individuals with potassium deficiency should always be checked for magnesium deficiency as well. It has been demonstrated that in the presence of magnesium depletion, it is very difficult to replete potassium stores. Some reports suggest the use of a calcium citrate source containing vitamin D to enhance calcium absorption. This is an effervescent formulation and it is taken as two tablets dissolved in a glass of cold water per day. Calcium citrate is reported to be better absorbed and utilized than calcium carbonate. The use of fluoride in a liquid formulation (potassium fluoride 180 mg/cc) has shown to help bind calcium in the bones. Fluoride drops are taken as 1 drop in juice or water three times per day. This is a prescription item and must be made up by the pharmacist. The dose can be slowly increased per the physician. Fluoride in this concentration can irritate the gastric lining. Slow increases in dose are reported to be important to prevent this side-effect.

Finally, zinc is important in wound healing. It also functions as an anti-oxidant. It is helpful in the treatment of acne. It hastens healing of peptic ulcer disease and burns. The recommended dose is currently 100 mg per day.

4. Nutrient Smell Test

The nutrient smell test, according to the invention disclosed herein, implements the olfactory system to test the appetite-satiety response to nutrients in order to compare and assess the nutrient loads, singularly or in comparison with each other, in the body.

Taste and smell are related both in functional process and feedback processes related to appetite and satiety. An important principle to consider is that the identity of a taste and its intensity are represented separately from its pleasantness. Thus, it is possible to represent what a taste is, and to learn about it even when we are not hungry. See, for example, Rolls, Chem. Senses 26: 595-604, (2001). In responding to taste as a measure of appetite or satiety, note that neuronal responsiveness declines as the subject reaches satiety. Accordingly, it is possible that a bad taste could actually represent over saturation or possibly toxicity. Indeed, after feeding to satiety, humans reported that the taste of the food on which they had been satiated tasted almost as intense as when they were hungry, though much less pleasant. See, for example, Rolls, et al., 1983).

Without being bound to any specific mechanism of action, one possible mechanism of action is that olfactory neurons in the primate orbitolfrontal cortex decrease their responses to a food eaten to satiety, but remain responsive to other foods, thus contributing to a mechanism for olfactory sensory-specific satiety. It has been shown in neuroimaging studies that the human orbitolfrontal cortex provides a representation of the pleasantness of odor in that the activation produced by the odor of a food eaten to satiety decreases relative to another food-related odor not eaten in the meal. In the same general area there is a representation of the pleasantness of the smell, taste and texture of a whole food, in that activation in this area decreases to a food eaten to satiety, but not to a food that has not been eaten in the meal. See, for example, Rolls, Chem. Senses (2001) supra. It is suggested that the body can indeed self-select nutrients tastefully even if those nutrients are mixed with other nutrients.

The nutrient smell test of the invention may vary the density, recipes and order of the nutrients for different insights into the dietary habits of the individual. The nutrients are smelled in an orderly, or an inorderly fashion. The order of sniffing the nutrients can be designed on the basis of several criteria, including, pH of the nutrients and the chemical reactivity of nutrients with respect to each other, among other factors.

The nutrient smell test allows an individual to test a collection of nutrients, including vitamins, minerals and essential oils in various recipes and combinations in order to elicit a positive response (appetite/desire) or negative response (satiety/no desire), score the response on a scale, for example, from desirable/appetite to not pleasant/satiety and record the scores to show changes in nutrient need.

The result of the test is recorded and can be used for future reference in order to assist the individual in future nutrient choices. The positive or negative response to each nutrient differs with each individual and signifies a pattern of nutrient's appetite (desire) or satiety (no desire) based upon his or her unique chemistry and dietary choices.

According to one embodiment, the smell test allows for 7 different responses, scored 1-7. Scores 1-3 represent an appetite (need), score 4 represent a neutral attraction, neither too much nor too little, (a take it or leave it sense) and a score of 5-7 represents satiety (no need), or no use at this time.

The test is simple and rapid. Each nutrient is assigned with a number. Starting with nutrient #1, the test taker smells the aroma-intensified nutrient to determine need and record the score. This determines which of the nutrients to take and how often to use the test. The scores can also lead the consumers to foods that provide the vitamins they most need.

The mean of the combined scores, for example for 20 nutrients, yields a statistical average of individual's nutrient storage. For example, a mean of 4 among all nutrients is equal to a full tank, but not much in reserve. A mean score below a 4 indicates that there is a shortage of available nutrients, and a mean score above a 4 indicates a reserve, or possible excess of nutrients. This is possibly due to the fact that all nutrients are interdependent and a lack or excess of some nutrients can prevent utilization or solubility of others. For example, the relationship of calcium to magnesium is well documented in this regard.

In general, a score of 4 is a neutral, pivot or a rest point. The score of 5 may actually be the first point of contact with no go. In electrical terms 4 may be a re-set point (e.g., open contact with no impedance or charge). That means the signal of 5 is the beginning of polarization into “No go”.

Standard deviation of the scores for the total of nutrients tested also yields information about the relationship of each nutrient to the other. Since nutrients are co-dependent and interrelated, a deficiency in one nutrient may prevent other nutrients from interacting properly or at full value to the body. This number indicates how capable the body of the individual is in adopting and responding to the internal and external changes related to physiological and environmental issues, respectively. The higher the standard deviation, the less biochemical energy is available in the bio-system at a moments notice and therefore less biochemical potential healing manpower. The lower the standard deviation, the more biochemical energy is available in the bio-system at a moments notice and therefore more biochemical potential healing manpower.

Variance indicates how often an individual's bio-system may flourish or fall into lack of biochemical energy, thereby giving an indication of overall health and stability of the body bio-system. Individuals demonstrating high variability (lots of numbers from high to low) have unpredictable biochemical energy and prone to chemical imbalances.

Also encompassed within the scope of the invention is the use of the smell test of the invention in veterinary animal medicine in order to detect nutritional imbalance in animals. The test is administered in animals such as, for example, mammals (e.g., dogs, cats, mice, rabbit, etc). The score system for animals is simple and is designed to demonstrate need or no need as represented by scores 1 and 2, respectively. An individual administers the test to the animal by releasing the aroma from the aroma-intensified nutrients. The animal would then smell the nutrient and according to the reaction of the animal to the aroma released from the nutrient, the individual test administerer scores the smell as either 1 or 2. The score indicates an imbalance of nutrients, and special need and/or surplus for certain food in the animal diet.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. The contents of all references, patents and published patent applications cited throughout this application are expressly incorporated herein by reference.

### EXAMPLES

### Example 1

### Nutrient Test For Individual Consumers

The smell of various nutrients was tested on individual consumers. The nutrients have been micro-encapsulated and coated on one or several pages of a book via scratch and sniff technology. Each nutrient was designated with a number, and a score system of 1-7 was designated for translation of smell to numerical values. Individual test takers scratched and sniffed each nutrient unanimously and scorc their smell according to their olfactory perception of the aroma associated with the nutrient. Scores 1-7 represented the following order:

1. desirable

2. pleasant

3. no taste

4. taste something

5. taste something not so good

6. dislike

7. awful

The identity of each of the nutrients tested was designated at the end of the test. The nutrients and the designated numbers were: #1: Vitamin A, #2: Vitamin B-1, #3: Vitamin B-2, #4: Vitamin B-6, #5: Vitamin B-12, #6: Vitamin C, #7: Vitamin D, #8: Vitamin E, #9: Biotin, #10: Choline, #11: Calcium Lactate Pentahydrate, #12: Folic Acid, #13: Betaine Hydrochloride/Pepsin, #14: Inositol, #15: Ferrous Fumerate (Iron), #16: Magnesium, #17: Niacin, #18: ParaAmino Benzoic Acid (PABA), #19: Pantothenic Acid, #20: Ammonium Chloride, #21: Beta Carotene, #22: Fish oil.

A wide range of responses was found to each nutrient from those that taught the nutrient smelled desirable to those who taught it smelled awful. When those who taught a nutrient smelled desirable were administered the nutrient for a period of one week or more, the smell changed over time from desirable to not so good and dislike. It was concluded that nutrients that smelled desirable or pleasant were at a critically low concentration and those nutrients that smelled dislike or awful were present at a high concentration in the body of the individual. The elevated concentration of the nutrient was recorded as unhealthy concentration and an indication of that the body has an over supply of the nutrient, which is excessive and would causes health problems, such as kidney and liver malfunction, if additionally administered.

### Example 2

### Smell Test for Minerals

Aroma associated with various minerals, either purified or as a food component, was tested on individual consumers. The minerals were micro-encapsulated on one or more pages of a stack of cards (e.g., single pin cards) and their smell were tested using scratch and sniff technology. Each mineral was designated with a number on the card, along with the score of 1-7 and the description of the scores as indicated in Example 1. Individual test takers have scratched and sniffed each nutrient unanimously. The test determines mineral deficiency using the smell test for 9 different minerals: 1. potassium phosphate, 2. zinc sulphate, 3. magnesium chloride, 4. copper sulfate, 5. potassium chromate, 6. potassium per-manganate 7. ammonium molybdate, 8. selenium selenite, and 9. potassium iodide.

Individual test takers smelled each mineral starting from mineral number 1 up to mineral number 9. Depending on the perceived smell of each mineral, scores of 1-7 were attributed. A score of 1 or 2 indicates deficiency with a 1 being quite deficient. A score of 3 indicates neutrality, while a score of 4 indicates sufficiency. If the smell sensation is neither pleasant nor disturbing but is clearly some smell, it is a 4, and indicates a lack of need at this time (4 is the ultimate goal). A score of 5, while not unpleasant, could be avoided, while a score of 6 or 7 indicates an excess of that mineral and should be avoided at this time.

In one individual, the score of 1, 2, 4, 5, 6, and 7 were non-existent for the first few months and all they obtained was a score of 3. After the first few months, they obtained a 4 on magnesium. Gradually, these individuals experienced scores of 1, 2, and 5 on various minerals, with an occasional 6 and 7. In another individual, the score for magnesium elevated up to 2 or even a 1 when the individual had a cold or some other ailments. After about a week or two, when the body was healed, the score for magnesium declined to a 4. The score of 4 is the pivot number—eat it or don't eat it, whereas a 3 is really a no signal. Accordingly, it is concluded that bodily need for magnesium is unpredictable and it is heavily influenced by minute changes in the biochemical and metabolic make up of individuals.

In certain individuals, after several months of smell test, a score of 4 was recorded for Mg and then on occasion a 1 or 2 and later a 5, and an occasional 6 on Zn, Mo and Se. Minerals smelled between 1 and 4 should be taken, together or individually, with liquids, such as, for example, and an acidic juice (orange, grapefruit, or pineapple), or ¼ tsp of vitamin C powder. The body requirement for the type and the concentration of minerals changes frequently, therefore frequent smell test of these minerals is important. Taking the minerals that is approved by the mineral smell test shifts the body into a balanced state which is the ultimate goal.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

What is claimed is:

1. A method of detecting an imbalance of nutrients in an individual comprising administration of a smell test, wherein the smell test comprises one or more support members and one or more nutrients that are aroma-intensified and incorporated into the one or more support members, the method comprising the steps of:
(a) listing nutrients and designating a number to each nutrient to create a nutrient list;
(b) designating a score system that allocates numerical values to describe a smell associated with each nutrient;
(c) smelling the nutrient to obtain a registered smell;
(d) allocating a score to the registered smell of the nutrient;
(e) identifying the nutrient by consulting the nutrient list of step (a); and
(f) tabulating and calculating numerical values obtained for each nutrient to obtain information on balance, surplus or deficiency of the one or more nutrients, Wherein, the administration of the smell test detects the imbalance of nutrients in the individual.

2. The method of claim 1, further comprising steps of:
(g) analyzing significance of results obtained in step (f); and
(h) extrapolating the results of the analysis to identify a specific pattern for long term or short term nutritional needs and counter-indication of specific nutrients and/or drugs for the individual.

3. The method of claim 1, wherein the one or more nutrients are aroma-intensified through a micro-encapsulation technique and step (c) is achieved through a scratch and sniff test.

4. The method of claim 1, wherein the one or more nutrients are aroma-intensified through a micro-encapsulation technique and step (c) is achieved through removing a strip from the one or more nutrients.

5. The method of claim 4, wherein the strip is part of the one or more support members, or is a separate entity.

6. The method of claim 1, wherein the one or more support members comprise post cards, books, magazines, single pin cards, scratch offs, pull tabs, card scan, game cards, phone cards, gift cards, and internet access cards, among others.

7. The method of claim 4, wherein the books and magazines are related to children, health, fashion, sport, cars, house decoration, food, fiction, and entertainment, among others.

8. The method of claim 4, wherein the book comprises a punched hole book, a ring type book, a bound book, and an address book, among others.

9. The method of claim 1, wherein the one or more support members comprise an internet and the aroma is intensified electronically.

10. The method of claim 1, wherein the list of nutrients in step (a) is located in a removably covered answer key that correctly identifies each nutrient and corresponding number of the nutrient.

11. The method of claim 1, wherein steps (b) and (d) are provided in a label located adjacent to each of the nutrients.

12. The method of claim 1, wherein the nutrients comprise vitamins, precursors of vitamins, oils, minerals, enzymes, co-enzymes, amino acid, or a combination thereof, among others.

13. The method of claim 1, wherein the smelling in step (c) is conducted on a pre-determined order of the nutrients.

14. The method of claim 13, wherein the pre-determined order is selected on the basis of chemical structure of the nutrient, pH of the nutrient, intensity of aroma associated with the nutrient, general type of aroma associated with the nutrient, or a combination thereof.

15. The method of claim 1, wherein the steps are conducted in an orderly or an inorderly fashion.

16. The method of claim 1, wherein the nutritional information is provided for general well being, diagnosis, treatment and/or prevention of a disease or disorder in the individual.

17. A smell test kit for detection of a deficiency or surplus of one or more nutrients in an individual via the olfactory system of the individual comprising on or more support members and a plurality of aroma-intensified nutrients incorporated into the one or more support members, the smell test kit further comprising:
(a) a nutrient list listing the one or more nutrients on a numerical order;
(b) a table that allocates numerical values to a description of aroma perceived by a test taker after the one or more nutrients was smelled;
(c) an instruction to use the test; and
(d) an explanation of the results of the test.

18. The smell test kit of claim 17, additionally comprising:
(e) a first section for statistical analysis of significance of the results; and
(f) a second section for extrapolating the results of the statistical analysis to identify a specific pattern for long term or short term nutritional needs and counter-indication of specific nutrients and/or drugs for the individual.

19. The test kit of claim 17, wherein the aroma intensified nutrients are micro-encapsulated and nutrients are smelled through scratch and sniff, or removal of a strip from the micro-encapsulated nutrient, or a combination thereof.

20. A nutritional analysis and recommendation book containing a nutrient smell test comprising:
(a) a multiplicity of aroma-intensified nutrients micro-encapsulated into the book; wherein an aroma associated with the nutrients is detectable via olfactory system of an individual test taker through a scratch and sniff test or a removal of an adhesive tape from the nutrients;
(b) a chart that designates a number for each of the nutrients;
(c) a table that allocates numerical values to a description of aroma perceived by the individual test taker after the nutrient was smelled;
(d) a first section for tabulating and calculating numerical values obtained in step (c) in order to obtain individualized nutritional recommendations;
(e) a second section that provides analysis of significance of results obtained by the individual test taker; and
(f) a third section that provides long term and short term nutritional recommendations and counter-indication advice on nutrients or drugs on the basis of results obtained in the first and the section sections.
